(19) United States
(12) Patent Application Publication
(10) Pub. No.: US 2025/0213298 A1
(43) Pub. Date: Jul. 3, 2025

(54) SHEATH WITH INTEGRATED SIGNAL LAYER

(71) Applicant: Boston Scientific Medical Device Limited, Galway (IE)

(72) Inventors: Jackie Leung, Richmond Hill (CA); Matthew MacDonald, Calgary (CA); Prateek Mathur, Toronto (CA); Miles Montgomery, Holland Landing (CA); Doran Avivi, Toronto (CA); Brian Ma, Etobicoke (CA); Esme Bonnell, Toronto (CA)

(21) Appl. No.: 19/002,370

(22) Filed: Dec. 26, 2024

Related U.S. Application Data
(60) Provisional application No. 63/616,372, filed on Dec. 29, 2023.

(51) Int. Cl. A61B 18/14; A61B 17/00; A61B 18/00; A61B 18/16

(52) U.S. Cl. CPC A61B 18/1492; A61B 2017/00526; A61B 2018/00077; A61B 2018/00083; A61B 2018/00351; A61B 2018/00577; A61B 2018/00839; A61B 2018/162

(57) ABSTRACT

Sheaths and method of manufacturing sheaths are disclosed. The sheath includes an elongated shaft defining a major lumen along a longitudinal axis and having a distal section opposite a proximal section. The shaft including a signal layer. The signal layer includes an electrically insulative substrate, an electrically conductive lead trace disposed on the electrically insulative substrate, and an interface pad exposed on a radially outward surface of the signal layer in the distal section and electrically coupled to the lead trace. The lead trace extends in a curvilinear serpentine route from the proximal section to the distal section. An electrode is exposed on the shaft and electrically coupled to the interface pad.

### CROSS REFERENCE TO RELATED APPLICATIONS

This application claims priority to U.S. Provisional Patent Application No. 63/616,372 entitled “SHEATH WITH INTEGRATED SIGNAL LAYER,” filed Dec. 29, 2023, which is hereby incorporated by reference in its entirety.

### TECHNICAL FIELD

The present disclosure relates to medical devices for catheterization procedures, such as medical devices for electrophysiological procedures. More specifically, the present disclosure relates to catheter elements, such as sheaths, and methods for manufacturing sheaths.

### BACKGROUND

Various medical fields use different types of catheters to achieve access to a physiological site in medical procedures. For instance, electrophysiological procedures involve guiding catheter elements into the heart and tracking the location of the catheter elements with respect to the heart. Catheter ablation is minimally invasive electrophysiological procedure to treat a variety of heart conditions such as supraventricular and ventricular arrhythmia. Example catheter elements used in catheter ablation can include mapping catheters, ablation catheters, guiding sheaths, dilators, and other medical tools, which can be referred to as catheter elements in this disclosure. Electrophysiological procedures can involve the visualization of the heart, heart activity, and the position of the catheter elements within the heart. A common visualization system involves the use of fluoroscopy, which can expose the patient and clinician to ionizing radiation. Electroanatomical mapping is an alternative visualization technique that does not involve the use of radiation.

Electroanatomical mapping allows a clinician to accurately determine the location of an arrhythmia, define cardiac geometry in three dimensions, delineate areas of anatomic interest, and permits imaging of the catheter elements for positioning and manipulation. Catheter elements used with electroanatomical mapping systems can include tracking capabilities, such as navigation enabled or impedance-based tracking methodologies. Navigation-enabled catheters use magnetic sensors in the presence of magnetic fields to track the location and orientation of the catheters. But not all catheter elements include magnetic sensors. Impedance-based catheter elements, such as sheaths use electrodes in the presence of electric fields to track the sheaths.

### SUMMARY

In Example 1, a sheath, comprising: an elongated shaft defining a major lumen along a longitudinal axis and having a distal section opposite a proximal section, the shaft including a signal layer; the signal layer having an electrically insulative substrate, an electrically conductive lead trace disposed on the electrically insulative substrate, and an interface pad exposed on a radially outward surface of the signal layer in the distal section and electrically coupled to the lead trace, the lead trace extending from the proximal section to the distal section; and an electrode exposed on the shaft and electrically coupled to the interface pad.

In Example 2, the sheath of Example 1, wherein the shaft further includes a spacer disposed around the interface pad and between the interface pad and the electrode.

In Example 3, the sheath of Example 2, wherein the spacer is an electrically insulative spacer.

In Example 4, the sheath of any of Examples 2 and 3, wherein the lead trace extends in a curvilinear serpentine route.

In Example 5, the sheath of any of Examples 2-4, wherein the spacer includes ribs.

In Example 6, the sheath of Example 1, wherein the shaft includes an elongated braided support member disposed between the signal layer and the electrode.

In Example 7, the sheath of Example 6, wherein the braided support member includes conductive fibers.

In Example 8, the sheath of Example 1, wherein the shaft includes an elongated and electrically insulative liner layer disposed radially underneath the signal layer, the liner layer having an inner wall defining the major lumen.

In Example 9, the sheath of Example 1, wherein the lead trace is disposed from the proximal section to the distal section along a radially inward surface of the signal layer.

In Example 10, the sheath of Example 4, wherein the curvilinear serpentine route is in a horseshoe pattern.

In Example 11, the sheath of any of Examples 9 and 10, wherein the lead trace is copper tracing laminated on the substrate.

In Example 12, the sheath of Example 1, wherein the substrate is a flexible and stretchable thermoplastic polyurethane.

In Example 13, the sheath of Example 1, wherein the electrode is electrically coupled to the interface pad via a conductive ink.

In Example 14, the sheath of Example 13, wherein the electrode is electrically coupled to an electrically conductive spring disposed in the conductive ink.

In Example 15, the sheath of Example 1, wherein the sheath includes a dilator.

In Example 16, a sheath, comprising: an elongated shaft defining a major lumen along a longitudinal axis and having a distal section opposite a proximal section, the shaft including a signal layer; the signal layer having an electrically insulative substrate, an electrically conductive lead trace disposed on the electrically insulative substrate, and an interface pad exposed on a radially outward surface of the signal layer in the distal section and electrically coupled to the lead trace, the lead trace extending in a curvilinear route from the proximal section to the distal section; and an electrode exposed on the shaft and electrically coupled to the interface pad.

In Example 17, the sheath of Example 16, wherein the shaft further includes a spacer disposed around the interface pad and between the interface pad and the electrode.

In Example 18, the sheath of Example 17, wherein the spacer is an electrically insulative spacer.

In Example 19, the sheath of Example 17, wherein the curvilinear route is serpentine.

In Example 20, the sheath of Example 17, wherein the spacer includes ribs.

In Example 21, the sheath of Example 16, wherein the shaft includes an elongated braided support member disposed between the signal layer and the electrode.

In Example 22, the sheath of Example 21, wherein the braided support member includes conductive fibers.

In Example 23, the sheath of Example 16, wherein the shaft includes an elongated and electrically insulative liner layer disposed radially underneath the signal layer, the liner layer having an inner wall defining the major lumen.

In Example 24, the sheath of Example 16, wherein the lead trace is disposed from the proximal section to the distal section along a radially inward surface of the signal layer.

In Example 25, the sheath of Example 24, wherein the curvilinear route is in a horseshoe pattern.

In Example 26, the sheath of Example 24, wherein the lead trace is copper tracing laminated on the substrate.

In Example 27, the sheath of Example 16, wherein the substrate is a flexible and stretchable thermoplastic polyurethane.

In Example 28, the sheath of Example 16, wherein the electrode is electrically coupled to the interface pad via a conductive ink.

In Example 29, the sheath of Example 28, wherein the electrode is electrically coupled to an electrically conductive spring disposed in the conductive ink.

In Example 30, the sheath of Example 16, wherein the sheath includes a dilator.

In Example 31, the sheath of Example 16, wherein the electrode is integrally formed with the interface pad.

In Example 32, a sheath, comprising: an elongated shaft defining a major lumen along a longitudinal axis and having a distal section opposite a proximal section, the shaft including a braided support member and a signal layer; the signal layer having an electrically insulative substrate, an electrically conductive lead trace disposed on the electrically insulative substrate, and an interface pad exposed on a radially outward surface of the signal layer in the distal section and electrically coupled to the lead trace, the lead trace extending in a curvilinear serpentine route from the proximal section to the distal section, the signal layer radially underneath the braided support member; and an electrode radially above the braided support member, exposed on the shaft and electrically coupled to the interface pad.

In Example 33, the sheath of Example 32, wherein the shaft includes an elongated and electrically insulative liner layer disposed radially underneath the signal layer, the liner layer having an inner wall defining the major lumen.

In Example 34, a method of forming a sheath, the method comprising: forming a signal layer having an electrically insulative substrate, an electrically conductive lead trace disposed on the electrically insulative substrate, and an interface pad exposed on a radially outward surface of the signal layer and electrically coupled to the lead trace, the lead trace extending in a curvilinear serpentine route from the proximal section to the distal section; forming an elongated shaft defining a major lumen along a longitudinal axis and having a distal section opposite a proximal section, the shaft including a braided support member and the signal layer; and electrically coupling an electrode to the interface pad, the electrode exposed on the distal section of the shaft.

In Example 35, the method of Example 34, and further including attaching a spacer disposed around the interface pad and between the interface pad and the electrode.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, the example clinical setting having an example electrophysiology system.

FIG. 2 is a schematic diagram illustrating an example sheath constructed according to the present disclosure that can be used with the example electrophysiology system. of FIG. 1.

FIG. 3A is a schematic diagram illustrating a first plan view from a first side of an example signal layer of the example sheath of FIG. 2.

FIG. 3B is a schematic diagram illustrating a second plan view from a second side, opposite the first side, of the example signal layer of FIG. 3A.

FIG. 3C is a schematic diagram illustrating a side view of the example signal layer of FIGS. 3A and 3B.

FIG. 3D is a schematic diagram illustrating a side view of another example signal layer of another example sheath constructed according to the present disclosure that can be used with the example electrophysiology system. of FIG. 1.

FIG. 4 is a schematic diagram illustrating a cross section view of a portion of the example sheath of FIG. 2.

FIGS. 5A-5C are schematic diagrams illustrating cross section views of other example sheaths constructed according to the present disclosure that can be used with the example electrophysiology system. of FIG. 1.

FIG. 6A illustrates a perspective view of a first example spacer member for use with the example sheath of FIG. 2.

FIG. 6B illustrates a perspective view of a second example spacer member for use with the example sheath of FIG. 2.

FIGS. 7A-7D illustrates side views of portions of an example shaft of the sheath of FIG. 2 during manufacturing.

FIG. 8 is a schematic diagram illustrating a cross section view of a portion of the example sheath of FIG. 2.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

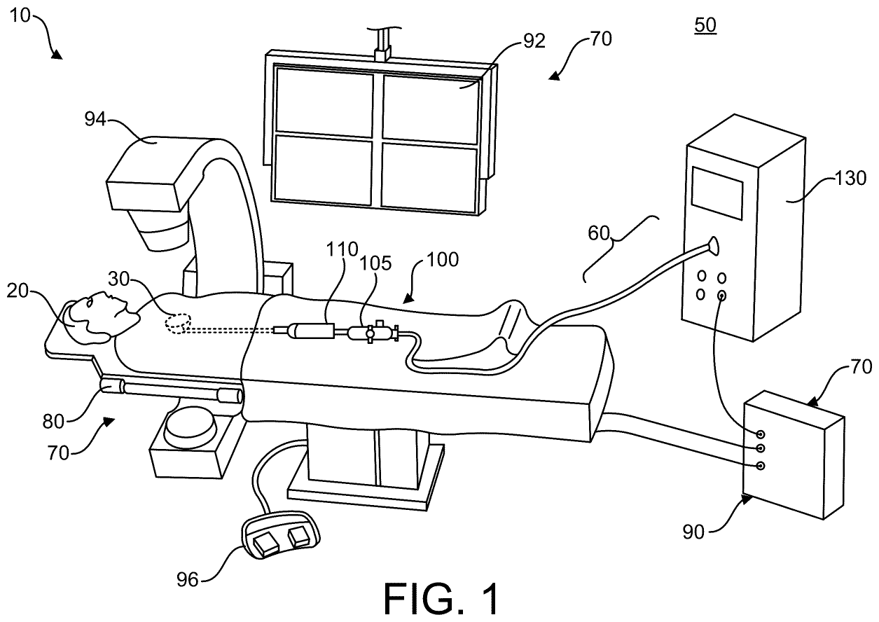

FIG. 1 illustrates an example clinical setting 10 for treating a patient 20, such as for treating a heart 30 of the patient 20, using an electrophysiology system 50, in accordance with the disclosure. The electrophysiology system 50 includes a catheter system 60 and an electroanatomical mapping (EAM) system 70. The example catheter system 60 includes an elongated catheter assembly 100, which in the example includes an ablation catheter 105 and a catheter sheath 110, and an electroporation console 130. Additionally, the catheter system 60 includes various connecting elements, such as cables, that operably connect the components of the catheter system 60 to one another and to the components of the EAM system 70. In general, the EAM system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The sheath 110 is operable to provide a delivery conduit through which the catheter 105 can be deployed to the specific target sites within the patient's heart 30. Access to the patient's heart can be obtained through a vessel, such as a peripheral artery or vein. Once access to the vessel is obtained, the catheter 105 can be navigated to within the patient's heart, such as within a chamber of the heart.

The example catheter system 60 is configured to deliver ablation energy to targeted tissue in the patient's heart 30 to create cell death in tissue, for example, rendering the tissue incapable of conducting electrical signals. An elongated catheter assembly, such as catheter assembly 100, can include a plurality of coaxially disposed catheter elements. For instance, a catheter defines a longitudinal axis that passes through a centroid of a cross section of the catheter element, such as the centroid of a cross section of a shaft of catheter 105 or a centroid of a cross section of a main lumen of a sheath 110. In the example, the catheter 105 is disposed within the sheath 110. The catheter 105 and sheath 110 are movable with respect to each other along the longitudinal axis.

The example catheter 105 includes an elongated catheter shaft and distal end configured to be deployed proximate to the target tissue, such as within a chamber of the patient's heart. The distal end may include a basket, balloon, spline, configured tip, or other deployment mechanism to effect treatment. The deployment mechanism can include an electrode assembly or array having a plurality of ablation electrodes. Each of the plurality of ablation electrodes is electrically coupled to a corresponding elongated lead conductor that extends along the shaft to a catheter proximal end. The lead conductors can be electrically coupled to a plug in the proximal region of the catheter 105, such as a plug configured to be mechanically and electrically coupled to the console 130, for example, either directly or via intermediary electrical conductors such as cabling. In one example, the console 130 is configured to provide an electrical signal, such as a plurality of concurrent or space-apart-time electrical signals, to the electrically connected catheter 105 along lead conductors to the spaced-apart electrodes to effect ablation.

The console 130 is configured to control aspects of the catheter system 60. The console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. The memory can be part of the one or more controllers, processors, or computers, or part of memory device accessible through a computer network. Examples of computer networks include a local area network, a wide area network, and the internet.

The EAM system 70 can be operable to track the location of the various components of the catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative embodiment, the EAM system 70 can include the OPAL™ HDx mapping system marketed by Boston Scientific Corporation. The mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 can generate a localization field, via the magnetic field generator 80, to define a localization volume about the heart 30, and a location sensor or sensing element on a tracked device, such as sensors on the electroporation catheter 105, generate an output that can be processed by the mapping and navigation controller 90 to track the location and orientation of the sensor or sensors, and consequently, the corresponding device, within the localization volume. In the illustrated embodiment, the device tracking is accomplished using magnetic tracking techniques, in which the field generator 80 is a magnetic field generator that generates a magnetic field defining the localization volume, and location sensors on the tracked devices are magnetic field sensors.

In other embodiments, impedance tracking methodologies may be employed to track the locations of the various devices. In such examples, the localization field is a set of independently oriented and spatially varying electric fields generated, for example, by an external field generator arrangement, such as surface electrodes, by intra-body or intra-cardiac devices, such as an intracardiac catheter and associated sheath 110, or both. In these examples, the location sensing elements can constitute tracking electrodes on the tracked sheath that generate outputs received and processed by the mapping and navigation controller 90 to track the location of the various location sensing electrodes within the localization volume. For instance, an impedance tracking methodology can employ the use of a patch electrode (not show) attached to the patient's body, a current or impedance based can be determined between the tracking electrode on the catheter and the patch electrode and the sheath and the patch electrode.

The EAM system 70 can be equipped for magnetic tracking capabilities, impedance tracking capabilities, or for both magnetic and impedance tracking capabilities. Regardless of the tracking methodology employed, the EAM system 70 utilizes the location information for the various tracked devices, along with cardiac electrical activity acquired by, for example, the electroporation catheter 105 or another catheter, sheath or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

In the case of impedance-based tracking with the EAM system 70, the catheters and sheaths include tracking electrodes disposed on the deflectable portions of the respective shafts. Multiple tracking electrodes can be employed on the deflectable portions of the catheters and sheaths for the EAM system 70 to detect and recreate the curvature of the catheters in the body. In one example, each tracking electrode is coupled to a corresponding lead conductor, or lead wire, which extends along the shaft to the proximal portion where it is coupled to an electrical connector. The electrical connector can be coupled to the EAM system 70 such as via cables.

Several constraints are employed in the design and implementation of tracking electrodes and associated lead conductors. Among these constraints include that each tracking electrode and associated lead conductor are to be electrically isolated from one another as well as from other conductive material in the sheath such as a conductive braided member along the length of the shaft. Additionally, precise placement of the tracking electrode on the shaft is desired. For instance, the tracking electrode can be radiopaque or echogenic, and clinicians can use the tracking electrode to visualize placement of the sheath while using fluoroscopy or ultrasound. Also, electrode location with respect to the catheter elements and interelectrode spacing are programmed parameters in several tracking and mapping software programs, and three-dimensional reconstruction and modeling is performed with electrode spacing as a constraint in the modeling curve. Thus, the design and implementation of sheath using tracking electrodes include lumens with ready access to carry conductor leads.

Sheath in electrophysiological procedures primary introduce and guide other catheters to within the anatomy. Unlike many diagnostic or ablation catheters used in electrophysiological procedures, sheaths include a large portion of the diameter taken up with a major lumen suitable for receiving diagnostic catheters, ablation catheters and other tools. Typical sheaths incorporate minor lumens along the shaft to carry wires that connect to the electrodes. The lumens are often disposed below or above braided regions of the shaft, which increase components and add to the bulk of the wall of the shaft or increase manufacturing complexity. The minor lumens can affect the bending and other mechanical properties of the shaft as well as the outer diameter of the shaft, which are limited via anatomical constraints.

With increasing number of electrodes as well as other electrical devices disposed at the distal portion of the shaft, a challenge exists to maintain desirable mechanical properties as well as a limited outer diameter of the shaft. Attempts to address these issues have removed the minor lumens from the shaft and the wires or leads are firmly affixed to the braided member, cover member, or a combination of both. Issues with these approaches include that the wires or leads provide little to no degrees of freedom for bending of the shaft, which creates problems in actuating steering mechanisms. Further, the wires and leads are under high stress during bending of the shaft, which increases the likelihood that the leads will break during a procedure, and the visualization of the distal tip will be lost.

This disclosure is directed to catheter elements, such as sheaths, and methods for assembling sheaths that include an elongated shaft defining a major lumen along a longitudinal axis and having a distal section opposite a proximal section. The shaft includes layers such as a cover member, liner layer, or a support member. The layers of the shaft also includes a signal layer. The signal layer includes an electrically insulative substrate, an electrically conductive lead trace disposed on the electrically insulative substrate, and an interface pad exposed on a radially outward surface of the signal layer in the distal section. The interface pad electrically coupled through the substrate to the lead trace. The lead trace extends in a curvilinear serpentine route from the proximal section to the distal section. An electrode is exposed on the shaft and electrically coupled to the interface pad. In one example, shaft includes a spacer disposed around the interface pad and between the interface pad and the electrode. In one embodiment, the substrate is a flexible, stretchable material, and the curvilinear serpentine route of the lead trace allows for bending and steering a reduced likelihood of breaking and affecting signal integrity. The signal layer is dedicated to transmitting the electrical signals and does not depend on overall composite design of the shaft, which allows for independent modification of signal pathways and number of electrodes without affecting the mechanical properties of the sheath.

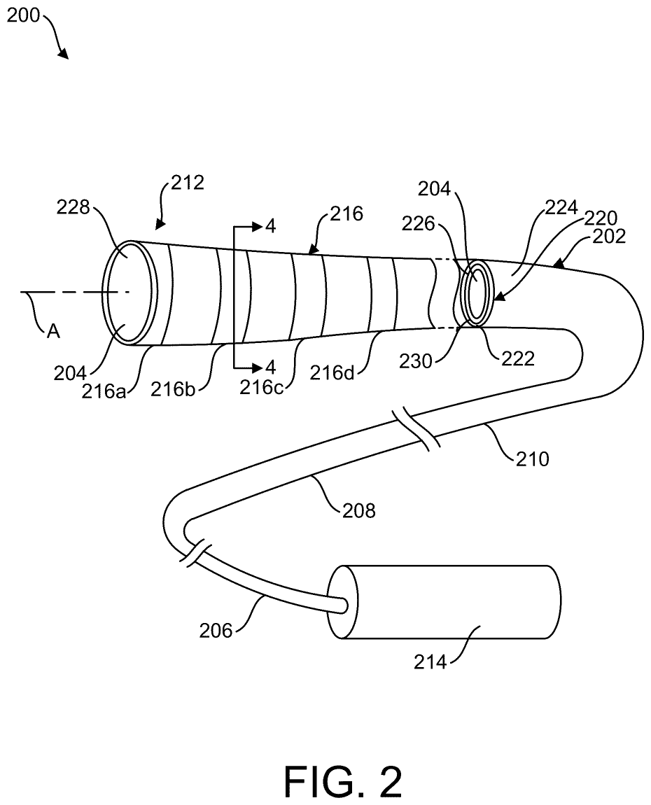

FIG. 2 illustrates an embodiment of a catheter element 200, such as a sheath, that can be used in the example clinical setting 10 in catheter assembly 100. For instance, sheath 200 can be further configured as a guide sheath, dilator, or other flexible, deflectable medical tool that can be tracked via an impedance tracking system such as EAM system 70. The sheath 200 includes an elongated shaft 202, such as an elongated and flexible shaft 202 defining a longitudinal axis A. The shaft 202 also defines a major lumen 204 along the longitudinal axis A and has a proximal section 206, an intermediate section 208, and a distal section 210 opposite the shaft 202 from the proximal section 206. The distal section 210 includes a distal tip section 212. The proximal section 206 can be coupled to a handle 214 proximal to the shaft 202. In some embodiments, the proximal section 206 or handle 214 can include a hemostatic valve that can be coupled to a source of irrigation fluid and a port to receive catheter elements within the major lumen 204. In some embodiments, the distal tip section 212 can be configured as a dilator tip.

The sheath 200 includes components disposed along the shaft 202. For instance, the sheath 200 includes components disposed on the distal section 210. In the illustrated embodiment, the sheath 200 includes a tracking electrode 216, such as a plurality of exposed tracking electrodes 216a . . . 216n, on the distal section 210 of the shaft 202. The tracking electrodes 216 are illustrated as ring electrodes mechanically coupled to the shaft 202. Each tracking electrode 216a . . . 216n is electrically coupled to a respective lead conductor extending along the shaft 202 and terminated at the proximal section 206. In the example, the tracking electrodes 216a . . . 216n are configured for use with an impedance-based tracking system to detect the position of the shaft 202, such as the distal tip section 212. In some embodiments, the tracking electrodes 216a . . . 216n are also radiopaque. In some embodiments, the distal section 210 can include other components disposed on or within the shaft 202, such as pull rings having steering wires disposed along minor lumens in the shaft extending to the proximal section 206 and various other sensors, such as a magnetic sensor, temperature sensor, a gyroscopic sensor, and an accelerometer within the distal section 210 of the shaft and coupled to leads disposed along the shaft and terminated at the proximal section 206. In some embodiments, the shaft 202 includes an irrigation conduit extending from the distal section 210 to the proximal section to supply an irrigation fluid to, for instance, the distal tip section 212. The proximal section 206 or the handle 214 in embodiments includes an electrical connection that can be coupled to an impedance-based tracking system, such as EAM system 70. In one example, the electrical connection is available under the trade designation LEMO. The proximal section 206 or the handle 214 in embodiments includes various controls to operate the steering wires and other connections to other sensors and provide fluid flow.

In the illustrated embodiment, the sheath 200 includes a plurality of concentric layers 220 disposed over all of or some of the shaft 202 and along longitudinal axis A, such as a cover member 222 forming an outer surface 224 of the sheath 200, and additional layers underneath the cover member 222 such as a support member 226, liner layer 228, and a signal layer 230, that extend along the shaft 202 and are coaxial with the major lumen 204. In some embodiments, the shaft does not include one or more of the cover member 222, support member 226, and liner layer 228. In various embodiments, the support member 226 is a flexible braid (e.g., stainless steel braid or a high-strength polymer braid, or braid formed from a combination of materials), a coiled shaft, a hypotube (e.g., a laser cut hypotube), or a high-density polyethylene (HDPE) tube. In some embodiments, the shaft 202 can include a liner layer 228 radially underneath the support member 226 and coaxial with the main lumen 204. In other embodiments, the liner layer 228 is the support member 226, and the shaft 202 does not include a braided member or other. The liner layer 228 can define an inner wall 234 of the main lumen 204. In examples, the liner layer 228 can be a thin wall constructed of polytetrafluoroethylene (PTFE). In some embodiments, the signal layer 230 or support member 226 includes features that form the inner wall 234 rather than the liner layer 228. In some embodiments, the cover member 230 can be formed as a coating of an electrically insulative reflowable plastic or thermoplastic material.

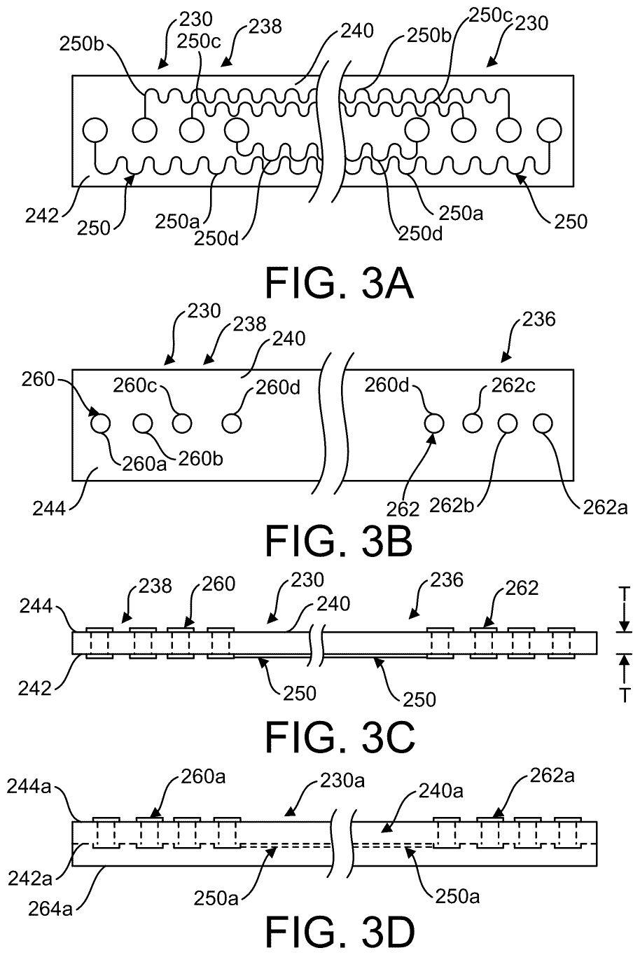

FIGS. 3A-3C illustrate an embodiment of the signal layer 230. The illustrated signal layer 230 includes a first end portion 236, configured to be at the proximal section 206 of the shaft 202 and a second end portion 238, configured to be at the distal section 210 of the shaft 202. The signal layer 230 includes an elongated, electrically-insulative substrate 240. The substrate 240 includes a first major surface 242, configured to be a radially inward surface of layers 220 on the shaft 202 and an opposite second major surface 244 configured to be a radially outward surface of layers 220 on the shaft 202. In one embodiment, the substrate 240 is flexible and is formed of an electrically insulative (nonconductive) material such as a thermoplastic. In another embodiment, the substrate 240 is both flexible and stretchable. In one example, the substrate is a thin layer of approximately 0.15 millimeters thickness along dimension T-T as illustrated in the side view of the signal layer 230 in FIG. 3C. An example of a suitable material for the substrate 240 is a polyether block amide or a thermoplastic polyurethane, or TPU.

The first major surface 242 of the substrate 240 is illustrated in in a first plan view FIG. 3A. The signal layer 230 further includes an electrically conductive lead trace 250, such as plurality of lead traces 250a . . . 250n, disposed on the first major surface 242. The lead traces 250a . . . 250n extend along first major surface 242 of the substrate 240 from the proximal end portion 236 to the distal end portion 238. The lead trace 250, or plurality of lead traces 250a . . . 250n, are formed as electrical traces configured to conduct an electrical signal along the shaft 202, such as from an electrical connection at the proximal portion 206 or the handle 214 to a corresponding electrode 216a . . . 216n. In one embodiment, the lead trace 250 is formed as a copper tracing laminated to the substrate 240. In the illustrated example, the lead trace 250 is configured to be on the radially inward surface of substrate 240 when formed as the signal layer 230 of the shaft 202.

The lead trace 250, or plurality of lead traces 250a . . . 250n, are formed to extend along the substrate in curvilinear serpentine route from the proximal end portion 236 to the distal end portion 238. In the illustrated example, the curvilinear serpentine route is in the configuration of a horseshoe pattern, or as an axially extending, alternating interconnected horseshoes. Other curvilinear serpentine routes are contemplated. The lead trace 250 configured in a curvilinear serpentine route provides flexibility when the shaft 202 is deflected during use and enables a robust electrical connection between the proximal section 206 and the corresponding electrode 216 over, for example, purely linear longitudinal traces. In one example, the curvilinear serpentine route extends along the entire longitudinal dimension of the lead trace 250. In another example, the curvilinear serpentine route extends along selected portions of the longitudinal dimension such as the portions subjected to the most deflection during use of the shaft 202. In still another example, the amount of curvilinear serpentine routing varies, such as higher pitched serpentine routing in areas of the shaft 202 subject to more deflection and lower pitched serpentine routing in areas of the shaft 202 subject to less deflection. In one example, a higher pitched serpentine routing includes narrow horseshoes, as measured along the longitudinal axis A, and a lower pitched serpentine routing includes wider horseshoes, as measured along the longitudinal axis A.

The second major surface 244 of the substrate 240 is illustrated in a second plan view FIG. 3B (opposite the first plan view of FIG. 3A). The signal layer 230 further includes an interface pad 260, such as interface pads 260a . . . 260n exposed on the second major surface 244 of the substrate 240, configured to be at the distal section 210 of the shaft 202. The interface pad 260 is electrically coupled through the substrate 240 to the lead trace 250. In the illustrated embodiment, the interface pads 260a . . . 260n are electrically coupled through the substrate 240 to a corresponding lead trace 250a . . . 250n. In the illustrated example, the interface pad 260 is configured to be on the radially outward surface of substrate 240 when formed as the signal layer 230 of the shaft 202. The interface pad 260 is configured to be electrically coupled to the electrode 216 such that the electrode 216 is in electrical communication with the lead trace 250. In another embodiment, the interface pad 260 is formed of a biocompatible material such as gold, and the electrode is integrally formed with the interface pad 260. The interface pad 260 is longitudinally positioned on the substrate 240 to align with the position of the electrode 216 on the shaft 202. For example, the interface pads 260a . . . 260n are each selectively longitudinally positioned on the substrate 240 to align with the position of the corresponding electrode 216 on the shaft 202. In some embodiments, the signal layer 230 further includes a proximal interface pad 262, such as proximal interface pads 262a . . . 262n exposed on the second major surface 244 of the substrate 240, configured to be at the proximal section 206 of the shaft 202. The proximal interface pads 262a . . . 262n are configured to be electrically coupled to an electrical connection in the proximal section 206 or the handle 214.

The signal layer 230 is presented as providing an electrical connection between the electrode 212 and the proximal section 206 or handle 214. In other embodiments, the signal layer 230 provide an electrical connection between other components on the distal section 210 of the shaft 202, such as sensors, and the proximal section 206 or handle. For example, the interface pad 260 can be electrically coupled to sensors on the distal section 210 of the shaft 202. In still other embodiments, other components can be laminated to the substrate and directly coupled to the interface pad 260 or to the lead trace 250. The number of lead traces 250a . . . 250n corresponds with at least the number of electrodes 216a . . . 216n to be attached to the shaft 202. In one embodiment, the shaft 202 includes four longitudinally spaced-apart electrodes 216 and four associated lead traces 250. In another embodiment, the number of lead traces 250a . . . 250n corresponds with the number of electrodes 216a . . . 216n plus the number of other components electrically on the distal section 210 coupled to the proximal section 206 or handle 214. In some embodiments, lead traces 250 can be provided onto substrate 240 via techniques of creating electrical circuits onto thermoset polymers or elastomers including photolithography via vapor deposition, advanced micro-electromechanical systems (MEMS) fabrication technology, flexible printed circuit board (PCB) techniques, and three-dimensional (3D) printing of conductive inks.

FIG. 3C illustrates the lead traces 250 are exposed on the substrate 240. In this embodiment, the signal layer 230 can be radially disposed on an electrically insulative layer 220 such as a liner layer 228 or a nonconductive support member 226. For instance, the radially inward surface of the signal layer 230 is disposed on a radially outward surface of the liner layer 228 or the radially inward surface of the support member 226. FIG. 3D illustrates another embodiment of the signal layer 230a in which an electrically insulative layer 264a, such as another TPU layer, is disposed on the lead trace 250a and first major surface 242a of the substrate 240a. The lead trace 250a is sandwiched between nonconductive insulative layer 264a and substrate 240a. Interface pads 260a are exposed on the second major surface 244a of the substrate 240a. The substrate 240a can also include proximal interface pads 262a. The signal layer 230a can be provided on a conductive surface such as on a radially outward surface of a conductive braided support member or otherwise exposed such as the insulative layer 264a forming the inner wall of the major lumen 204 in lieu of the liner layer 228.

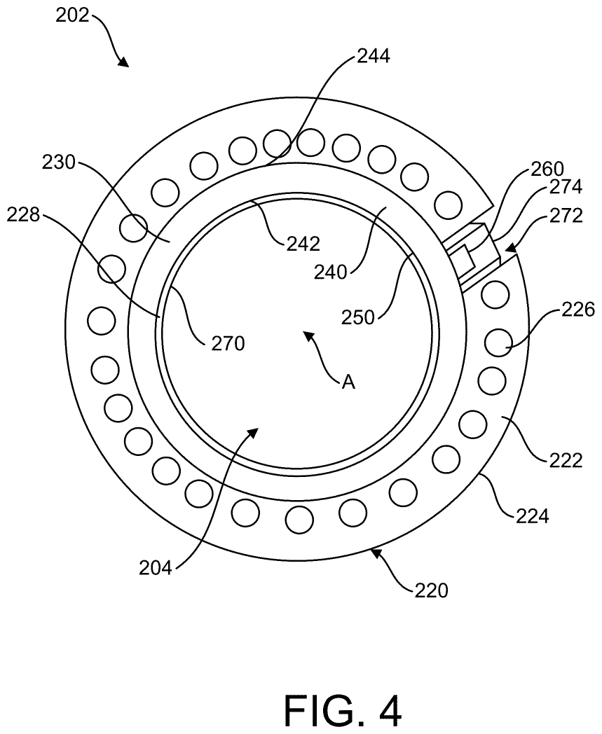

FIG. 4 illustrates a cross section of an embodiment of the shaft 202 taken laterally across the shaft 202 in the distal section 210 at lines 4-4. FIG. 4 illustrates an example of the layers 220 and does not show pull wire lumens or the electrode 216 for clarity. In the embodiment, the electrically insulative liner layer 228 is formed as the radially innermost layer and provides an inner wall 270 defining the major lumen 204. The signal layer 230 is disposed around the liner layer 228 and coaxial with the major lumen 204. As the liner layer 228 is constructed from an electrically insulative material such as PTFE, the radially inward surface of the signal layer 230, such as the first major surface 242, can include an exposed lead trace 250 against the liner layer 228 rather than a another electrically insulative layer, such as layer 264a. The TPU of the substrate 240 can bond to the liner layer 228. A support member 226 is disposed on the radially outward surface of the liner layer 230, such as the second major surface 244, and coaxial with the major lumen 204. In some examples, the support member 226 can include electrically conductive elements but is separated from the lead trace 250 via the electrically insulative substrate 240 of the signal layer 230. The cover member 222 is provided on the radially outward side of the support member 226 to form the outer surface 224.

The support member 226 is illustrated in the embodiment as a braided member 226, which can provide characteristics to the sheath 200 such as reduced kinking, wrinkling, or buckling of the shaft 202 and can provide enhanced balance for pushability, deflectability, and torque transmission such as during rotation about the longitudinal axis A. (In some embodiment, the signal layer 230 can also influence these properties.) In another embodiment, the support member 226 can include a coiled shaft member, a laser cut hypotube, or other elongated shaft material such as HDPE. In the illustration, the braided member as the support member 226 is constructed from a woven fabric or layer of braided strands or fibers that form interstitial spaces between the fibers. The braided member as the support member 226 can further be characterized by the warp and weft and bias of the fibers as well as picks per inch. For instance, in some embodiments, the picks per inch can remain generally uniform along the entire longitudinal length of the braided member as the support member 226. In some embodiments, the picks per inch can vary along portions of the longitudinal length of the braided member 226. The braided member as the support member 226 can be constructed from fibers that include stainless steel fibers, such as electrically conductive fibers, or high strength polymer fibers, or as layers of different materials. In some examples, the support member 226 can include more than one braided member, such as an innermost braided member and an outermost braided member.

The cover member 222, or outer layer in the embodiment, is disposed on the support member 226 and provides an outer surface 224. In some embodiments, the cover member 222 can be formed as a coating of an electrically insulative reflowable plastic or thermoplastic material that extends over the support member 226 and seals underlying components of the shaft 202. For instance, the coating can seep by reflowing over braided material of the support member 226 such as over the fibers and into the interstitial spaces of the braided material of the support member 226. In one example, the cover member 222 is a polyether block amide and, in some examples, is available under the trade designations PEBAX from Arkema, S.A., and VESTAMID E from Evonik Industries, AG. Electrodes 216 are exposed on the cover member 222.

In the illustrated example, a radially extending electrical passage 272 is formed in the shaft 202 between the outer surface 224 and the signal layer 230 to expose the interface pad 260. In one example, the electrical passage 272 is formed be spreading apart an interstitial space in the braided fabric of the support member 226. In another example, the electrical passage 272 is formed via drilling a hole through the support member 226 to the signal layer 230. In embodiments, an electrical passage 272a . . . 272n is formed in the shaft 202 corresponding with for each interface pad 260a . . . 260n associated with an electrode 216a . . . 216n. For example, the radially extending electrical passage 272 is formed at the location of the electrode 216 and the associated interface pad 262. The electrical passage 272 allows an electrical coupling between the electrode 216 on the outer surface 224 of the shaft 202 and the radially spaced apart interface pad 260 on the second major surface 244 of the signal layer 230. In some examples, an electrically conductive spring, wire, or electrically conductive filler can be provided in the electrical passage 272 to make a connection between the interface pad 260 and the electrode 216. In embodiments in which the support member 226 includes electrically conductive features, such as conductive fibers in the braided member, an electrically insulative spacer member is disposed along the sides of the electrical passage to electrically insulate an electrical connection between the interface pad 260 and electrode 216 from the electrically conductive support member 226.

In one embodiment, the liner layer 228 is formed over a manufacturing mandrel to create the major lumen 204. The signal layer 230 is wrapped, such as folded or coiled, over the liner layer 228 on the mandrel or the mandrel and attached, such as spot welded via heat or ultrasound or taped together. In order to remained fixed together, the signal layer 230 can include an overlapping region in which one longitudinally extending side is wrapped around a portion of the other longitudinally extending side to facilitate the bond and allow stretching of the signal layer 230. A PTFE liner layer 228 will bond to a TPU substrate 240 of the signal layer 230 or can be enhanced with a polyether block amide tie layer. The braided member 226 is braided over the signal layer 228 or, in the case of braid transfer, the braided member 226 is slid over the signal layer 228. In some examples, the braided member 226 holds the signal layer 230 in place without additional welding or taping. In other embodiments, the pull wire lumens can be incorporated in a manner known in the art, such as between the signal layer 230 and the support member 226 or between the liner layer 228 and the signal layer 230 in a similar manner as between the support member and the liner layer of typical shafts. The reflowed material will also bond to the TPU of the signal layer 230. The electrical passage 272 can be formed from the outer surface 224 to the second major surface 244 of the signal layer 230 to expose the interface pad 260. A spacer member 274 can be inserted into the electrical passage, and the electrode coupled to the associated interface pad.

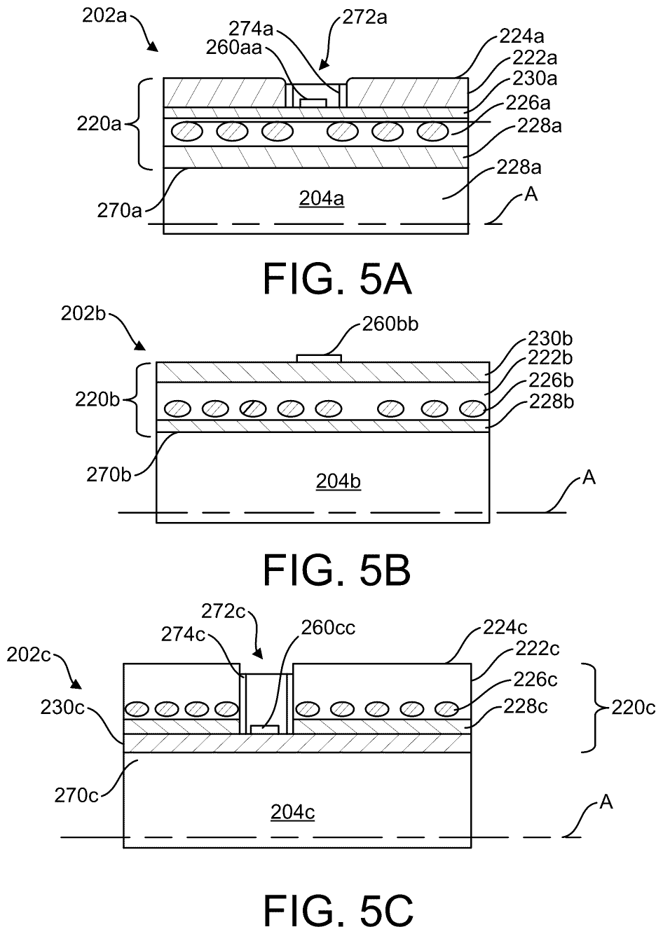

FIGS. 5A-5C illustrate cross sections of three of several alternative embodiments of the shaft 202 taken longitudinally along the respective distal sections of shafts 202a-202c. FIGS. 5A-5C do not show pull wire lumens or the electrode 216 for clarity.

FIG. 5A illustrates a portion of shaft 202a having a liner layer 228a forming an inner wall 270a defining the major lumen 204a, a braided support member 226a wound on the liner layer 228a and a signal layer 230a disposed on the braided support member 226a. In an example in which the braided support member 226a includes electrically conductive features, such as stainless-steel fibers, the signal layer 230a is provided with an electrically insulative radially innermost layer, such as insulative layer 264a illustrated in FIG. 3D, to electrically isolate conductive traces on the first major surface of the substrate from the support member 226a. An electrically insulative cover member 222a of reflowed plastic is disposed on the second major surface of signal layer 230a. Electrical passage 272a is formed in the cover member 222a to expose interface pad 260aa on the signal layer 230a. The electrical passage 272a can include a spacer member 274a for example to support the integrity of the shape of the electrical passage 272a. In the example, the spacer member 274a is not disposed against electrically conductive features in the layers 220a because the cover member 222a is electrically insulative, so that the spacer member 274a can be formed of an electrically conductive material as well as an electrically insulative material.

FIG. 5B illustrates a portion of a shaft 202b with layers 220b having liner layer 228b forming inner wall 270b defining a major lumen 204b, a braided support member 226b wound on the liner layer 228b and an electrically insulative cover member 222b disposed on the support member 226b. The signal layer 230b is disposed on the cover member 222b. Electrical traces on the first major surface of the substrate of the signal layer 230b are disposed against the electrically insulative cover member 222b. An electrode can be affixed directly to the interface pad 260bb on the second major surface of the signal layer 230b without an electrical passage formed in the shaft 202b. In some examples, the interface pad 260bb can provide the functions of the electrode, which is then optional. In some embodiments, the second major surface of the signal layer 230b forms the outer surface 224b. In other embodiments, a second cover member can be formed over the signal layer 230b.

FIG. 5C illustrates a portion of a shaft 202c with layers 220c having a signal layer 230c as the radially innermost layer 220c and forming the inner wall 270c defining the major lumen 204c. The signal layer 226c is provided with an electrically insulative radially innermost layer, such as insulative layer 260a illustrated in FIG. 3D, to electrically isolate, or otherwise protect, conductive traces on the first major surface of the substrate from the major lumen 204c. A liner layer 228c is formed on the second major surface of the signal layer 230c, a braided support member 226c is wound on the liner layer 228c, and a cover member 222c is disposed on the support member 226c to form the outer surface 224c. An electrical passage 272c is formed through the cover member 222c, support member 226c, and liner layer 228c to expose an interface pad 260cc on the signal layer 230c. A spacer member 274c is disposed against the layers 220c within the electrical passage 272c.

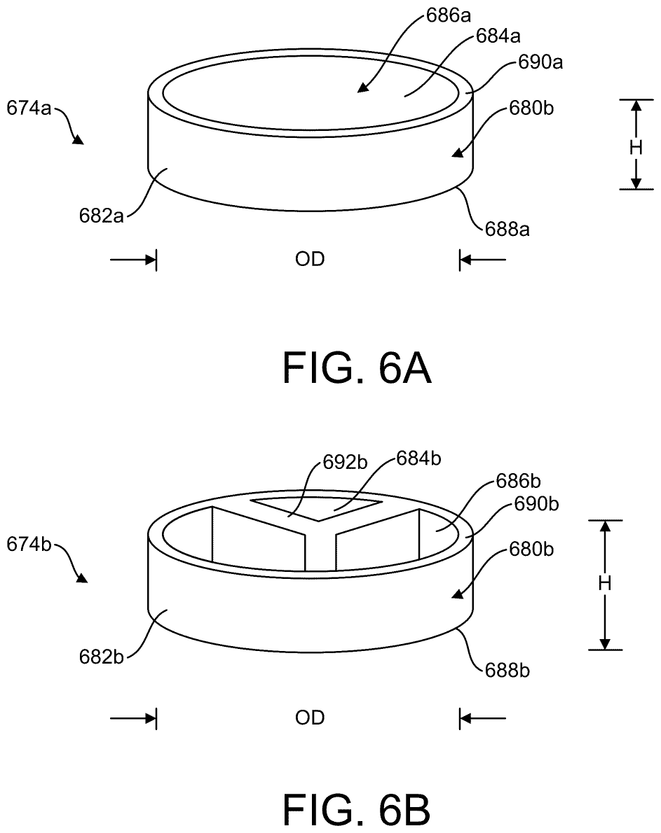

FIGS. 6A and 6B illustrate two examples of spacer members 674a, 674b configured to be disposed within an electrical passage on a shaft, such as spacer member 274 disposed in electrical passage 272 on shaft 202 in FIG. 4. Spacer members 674a, 674b include a wall, such as a cylindrically shaped wall 680a, 68b having an outer surface 682a, 682b and an inner surface 684a, 684b forming passageway 686a, 686b, a first end 688a, 688a and an opposite second end 690a, 690b. In one embodiment, the outer diameter OD of the spacer member 674a, 674b is approximately 2 millimeters and the height H, from the first end to the second end, of the spacer member 674a, 674b is approximately 0.2 to 0.5 millimeters. For instance, the wall 680a, 680b is configured to include the interface pad 262 within the inner surface 684a, 684b and passageway 686a, 686b and is strong enough to withstand forces on the outer surface 682a, 682b attempting to close the electrical passage 272 on the shaft 202, particularly from the fibers of the braided support member 226. The height H of the spacer member 674a, 674b in one embodiment is selected to extend from the signal layer 230 through the braided support member 226 and possibly into the cover member 222, but not to extend radially past the outer surface 224 of the shaft 202. In one example, spacer member 674b includes spokes or ribs 692 attached to the inner surface 684b to provide additional support the wall 680b from forces on the shaft 202 attempting to close the passageway 686b or collapse the wall 680b. The spacer members 674a, 674b can be constructed from an electrically insulative material for examples in which the outer surface 682a, 682b of spacer member 674a, 674b will contact an electrically conductive layer in the shaft 202, such as a stainless-steel braided support member 226, so as to electrically isolate the interface pad 260, electrode 216, and connector between the interface pad 262 and electrode 216 from the electrically conductive layer in the shaft 202. In other embodiments, the spacer member 674a, 674b can be constructed from an electrically conductive material for examples in which the outer surface 682a, 682b of spacer member 674a, 674b will not be in contact an electrically conductive layer in the shaft 202, such as if braided support member 226 is constructed from polymer fibers, and can enhance the electrically connection between the interface pad 260, electrode 216, and connector between the interface pad 260 and electrode 216.

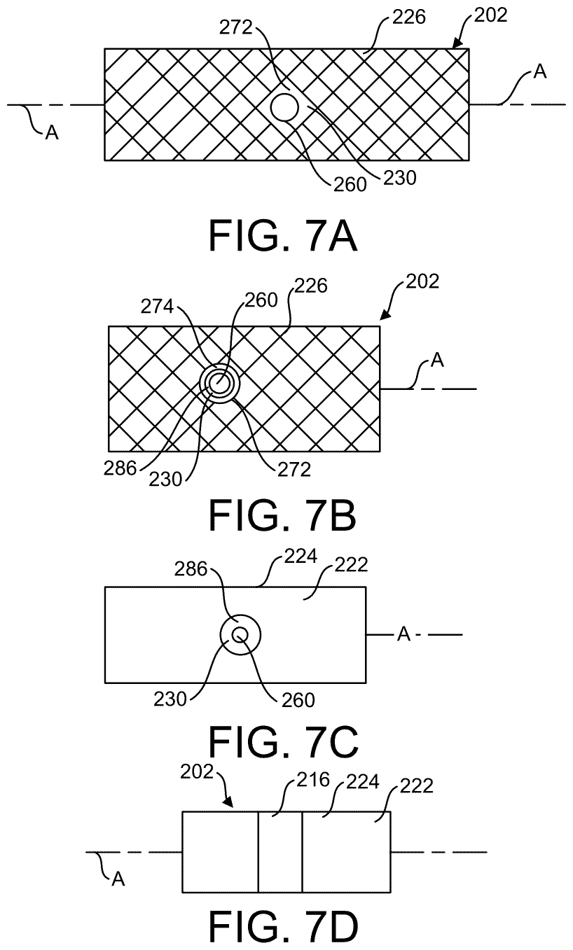

FIGS. 7A-7D illustrate a side view of a process of manufacturing the shaft 202 using a spacer member, such as cylindrical spacer member 274, such as spacer member 674a. the cylindrical configuration radially disposed on shaft 202. FIG. 7A illustrates a braided support member 226 radially over a signal layer 230 in which an interstitial space between the fibers of the support member 226 has been separated or opened to form an electrical passage 272. In one example, the support member 226 can be opened during the braiding process to create a pocket, such as via a variation braider. In another example, the support member is opened after braiding. A portion of the signal layer 230 is visible in the electrical passage 272 and an interface pad 260 is exposed on the signal layer 230. FIG. 7B illustrates spacer member 274 disposed within the electrical passage 272 and positioned such that the interface pad 260 is exposed within a passageway 286 of the spacer 274. The spacer member 274 keeps the electrical passage 272 open, or the spacer member 274 keeps the fibers of the support member 226 from closing the passageway 286. FIG. 7C illustrates a cover member 222 disposed on the shaft 202 covering the braided support member 226. Prior to applying the cover member 222, the spacer member 274 can be masked or covered so that the passageway 286 does not fill with reflow. FIG. 7D illustrates an electrode 216 disposed on the outer surface 224 of the shaft 202, over the cover member 222, which has been electrically connected to the interface pad 260 of the signal layer 230 and in an axial location over the passageway 286.

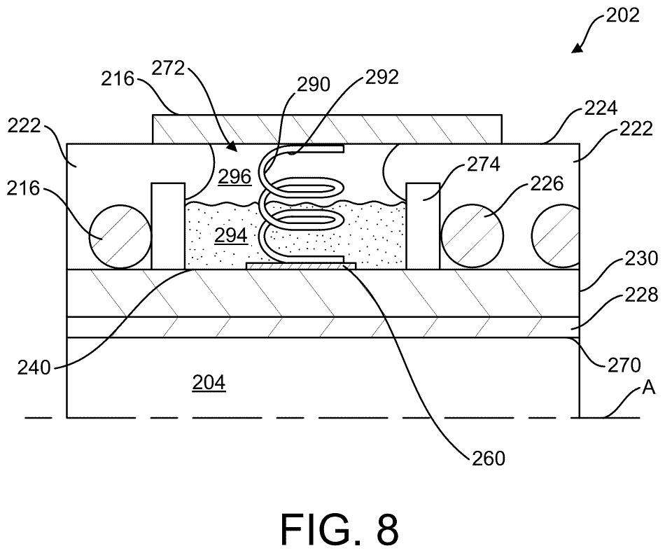

FIG. 8 illustrates a cross sectioned side view of a portion of the shaft 202 in which the interface pad 260 has been electrically coupled to the electrode 216. Shaft 202 includes signal layer 230 disposed radially on liner layer 228. The interface pad 260 is exposed on signal layer 230. The spacer member 274 is disposed around the interface pad 260 on the signal layer 230 and between the interface pad 260 and the electrode 216. The interface pad 260 is disposed within passageway 686 of the spacer member 274. The spacer member 274 urges apart fibers of the braided support member 226, which is disposed radially on the signal layer 230 and forms electrical passage 272. The cover member 222 is disposed on the support member 226, and the cover member 222 has reflowed through interstitial spaces between the fibers of the braided support member 226 to bond with the signal layer 230. The cover member 222 forms the outer surface 224 of the shaft 202 on which the electrode 216 is disposed radially above the electrical passage 272, the interface pad 260, and the spacer member 274. In the illustrated example, the fibers of the braided support member 226 are electrically conductive so that the spacer member 274 is electrically insulative. In other examples, the fibers of the braided support member 226 are electrically insulative so that the spacer member 274 can be electrically conductive or electrically insulative.

The interface pad 260 is electrically coupled to the electrode 216 via a wire 290. In the illustrated embodiment, the wire 290 is formed as a spring mechanism of slightly larger radial length than the distance between contact points on the interface pad 260 and the inner wall of the electrode 216. Accordingly, the spring wire 290 is loaded and urged toward the interface pad 260 and the inner wall of the electrode 216 to maintain the electrical connection. In one embodiment, the wire 290 is welded or soldered at spot 292 on the inner wall of the electrode 216 for a robust mechanical and electrical connection via an electrically conductive weld. Techniques such as welding and soldering the wire 290 to the interface pad 260, however, introduce heat that can damage the shaft 202 of a sheath catheter 200. To maintain the integrity of the electrical connection of the wire 290 and the interface pad 260 in the illustrated embodiment, a conductive adhesive 294 is disposed within the passageway 286 of the spacer member 274. The amount of conductive adhesive 294 is selected to remain within the spacer member 274 and not spill over onto the electrically conductive fibers of the braided support member 226. In another embodiment, a conductive ink is applied instead of the conductive adhesive 294 within the passageway 286 of the spacer member 274. In still another embodiment, an electrically insulative adhesive is applied instead of the conductive adhesive 294 within the passageway 286 of the spacer member 274. In the illustrated embodiment, the electrical passage 272 further includes an electrically insulative adhesive 296 over the conductive adhesive 294, which can fill and seal the electrical passage 272. In other embodiments, such as an electrically conductive spacer member 674a, 674b used in the shaft, the conductive adhesive 294 can fill the passage 686a, 686b.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A sheath, comprising:
an elongated shaft defining a major lumen along a longitudinal axis and having a distal section opposite a proximal section, the shaft including a signal layer;
the signal layer having an electrically insulative substrate, an electrically conductive lead trace disposed on the electrically insulative substrate, and an interface pad exposed on a radially outward surface of the signal layer in the distal section and electrically coupled to the lead trace, the lead trace extending in a curvilinear route from the proximal section to the distal section; and
an electrode exposed on the shaft and electrically coupled to the interface pad.

2. The sheath of claim 1, wherein the shaft further includes a spacer disposed around the interface pad and between the interface pad and the electrode.

3. The sheath of claim 2, wherein the spacer is an electrically insulative spacer.

4. The sheath of claim 2, wherein the curvilinear route is serpentine.

5. The sheath of claim 2, wherein the spacer includes ribs.

6. The sheath of claim 1, wherein the shaft includes an elongated braided support member disposed between the signal layer and the electrode.

7. The sheath of claim 6, wherein the braided support member includes conductive fibers.

8. The sheath of claim 1, wherein the shaft includes an elongated and electrically insulative liner layer disposed radially underneath the signal layer, the liner layer having an inner wall defining the major lumen.

9. The sheath of claim 1, wherein the lead trace is disposed from the proximal section to the distal section along a radially inward surface of the signal layer.

10. The sheath of claim 9, wherein the curvilinear route is in a horseshoe pattern.

11. The sheath of claim 9, wherein the lead trace is copper tracing laminated on the substrate.

12. The sheath of claim 1, wherein the substrate is a flexible and stretchable thermoplastic polyurethane.

13. The sheath of claim 1, wherein the electrode is electrically coupled to the interface pad via a conductive ink.

14. The sheath of claim 13, wherein the electrode is electrically coupled to an electrically conductive spring disposed in the conductive ink.

15. The sheath of claim 1, wherein the sheath includes a dilator.

16. The sheath of claim 1, wherein the electrode is integrally formed with the interface pad.

17. A sheath, comprising:
an elongated shaft defining a major lumen along a longitudinal axis and having a distal section opposite a proximal section, the shaft including a braided support member and a signal layer;
the signal layer having an electrically insulative substrate, an electrically conductive lead trace disposed on the electrically insulative substrate, and an interface pad exposed on a radially outward surface of the signal layer in the distal section and electrically coupled to the lead trace, the lead trace extending in a curvilinear serpentine route from the proximal section to the distal section, the signal layer radially underneath the braided support member; and
an electrode radially above the braided support member, exposed on the shaft, and electrically coupled to the interface pad.

18. The sheath of claim 17, wherein the shaft includes an elongated and electrically insulative liner layer disposed radially underneath the signal layer, the liner layer having an inner wall defining the major lumen.

19. A method for forming a sheath, the method comprising:
forming a signal layer having an electrically insulative substrate, an electrically conductive lead trace disposed on the electrically insulative substrate, and an interface pad exposed on a radially outward surface of the signal layer and electrically coupled to the lead trace, the lead trace extending in a curvilinear serpentine route from the proximal section to the distal section;
forming an elongated shaft defining a major lumen along a longitudinal axis and having a distal section opposite a proximal section, the shaft including a braided support member and the signal layer; and
electrically coupling an electrode to the interface pad, the electrode exposed on the distal section of the shaft.

20. The method of claim 19, and further including attaching a spacer disposed around the interface pad and between the interface pad and the electrode.
